(19) United States
(12) Patent Application Publication
(10) Pub. No.: US 2023/0404405 A1
(43) Pub. Date: Dec. 21, 2023

(54) Thermal Health Monitor

(71) Applicants: Nagesh Somayajula, Centennial, CO (US); Chinmay Somayajula, Centennial, CO (US); Nagalakshmi Somayajula, Centennial, CO (US)

(72) Inventors: Nagesh Somayajula, Centennial, CO (US); Chinmay Somayajula, Centennial, CO (US); Nagalakshmi Somayajula, Centennial, CO (US)

(21) Appl. No.: 18/217,977

(22) Filed: Jul. 3, 2023

(51) Int. Cl. A61B 5/00; A61B 5/11; G16H 80/00; G06T 7/00; A61B 5/01

(52) U.S. Cl. CPC A61B 5/0008; A61B 5/1117; A61B 5/747; G16H 80/00; G06T 7/0012; A61B 5/01; A61B 5/0033; G06T 2207/20081

(57) ABSTRACT

A home health monitoring system incorporates thermal cameras as detectors, enabling real-time monitoring and analysis of thermal data. The system monitors the activities and well-being of individuals or pets within its field of view. By detecting anomalies such as sudden falls, collapses, or medical emergencies through the identification of variations in body temperature or thermal patterns, the system can extract valuable insights about the activities and overall health of the monitored individuals or pets. It provides a comprehensive solution for real-time monitoring and analysis of thermal data. This information serves as the basis for generating timely reports, which are shared with nearby help centers to ensure swift and appropriate responses in emergencies. The integration of thermal imaging technology significantly enhances the effectiveness and accuracy of the home health monitoring system, thereby facilitating improved care and intervention for the monitored individuals or pets.

### BACKGROUND OF THE INVENTION

### Field of the Invention

The inventive subject matter relates generally to a home health monitoring system that utilizes thermal imaging technology to detect changes in an individual's body temperature and thermal patterns as an indicator of their health status.

### Description of Related Art

Existing home health monitoring systems mainly rely on non-thermal sensing modalities like motion sensors, contact sensors, and ambient sensors. While useful, these systems lack the capability to directly detect changes in skin temperature and heat distribution—both of which can serve as early indicators of potential health issues.

Some clinical thermal imaging systems have been developed to monitor patients' health conditions. However, these systems are designed for use within medical facilities and hospitals, not home environments. They also tend to focus on specific applications or health conditions, rather than providing comprehensive monitoring capabilities.

The inventive subject matter involves a home health monitoring system that employs a thermal camera as the primary sensor. By continuously capturing and analyzing thermal data from individuals within a home, the system can detect anomalies in body temperature and heat distribution patterns that suggest emerging health problems. The system then alerts designated contacts to facilitate timely intervention.

The integration of thermal imaging aims to significantly improve the sensitivity, accuracy, and responsiveness of health monitoring within home environments, as variations in skin temperature and thermal patterns often precede physiological changes that are detectable by conventional sensors. However, existing thermal imaging systems for healthcare monitoring have not been designed specifically for use in homes.

### BRIEF SUMMARY OF THE INVENTION

The present invention provides a comprehensive home health monitoring system utilizing thermal imaging technology. A thermal camera is installed in the home environment to continuously capture thermal images of individuals or pets within its field of view.

Thermal images are fed into a machine-learning model trained on both real and synthetic thermal data. The model detects and analyzes variations in body temperature, heat distribution patterns, and other thermal anomalies that may indicate potential health issues.

Through the machine learning model's ability to recognize abnormalities in thermal patterns, the system can identify situations like sudden falls, collapses, or medical emergencies. This allows the system to generate timely alerts that are reported to nearby hospitals, care facilities, family members, and friends.

The ability to detect and analyze thermal anomalies, combined with rapid alert generation and reporting, helps enable swift medical response and treatment when needed. Timely intervention facilitated by the system has the potential to save lives and mitigate health consequences by getting individuals the care they require as soon as possible.

The integration of machine learning and thermal imaging technology aims to provide a comprehensive, accurate, and proactive approach to health monitoring within home environments. By detecting subtle changes indicative of emerging health problems, the system seeks to optimize care, intervention, and outcomes for those it monitors.

### BRIEF DESCRIPTION OF DRAWINGS

FIG. 1 shows a visual representation of the intended setup for the Thermal Health Monitor (THM) camera device, highlighting its placement on a wall or stable mountable item, the monitoring field of view, and the Wi-Fi connectivity module.

FIG. 2 incorporates various components to enable advanced functionality and reliable operation. A Wi-Fi connection for seamless communication capabilities, allowing the device to interact with other devices and systems. The securely mounted thermal camera plays a crucial role in capturing and recording thermal data, providing valuable insights into temperature variations.

FIG. 3 outlines the process involved in thermal camera-based monitoring.

FIG. 4 outlines a comprehensive monitoring process that combines thermal imaging technology, AI analysis, and efficient communication protocols. It aims to swiftly identify emergency situations and health conditions within a monitored location, enabling timely notifications to medical personnel and generating SOS alerts for individuals in need. By establishing a seamless connection with emergency services, the system ensures that immediate assistance is provided to mitigate the impact of emergency scenarios and enhance overall safety.

FIG. 5 outlines a process for utilizing customized training data sets and a Single-Shot-Multibox Detector (SSD) for real-time detection. The system includes a trained data model and a mobile application designed to monitor and control a health monitor device. Real-time data is captured using a thermal camera and subsequently analyzed to detect health conditions, assess bacterial probabilities, and differentiate between subjects.

FIG. 6 presents a comprehensive and systematic approach that integrates image capture, health condition identification, decision-making, continuous monitoring, alert management, and emergency response within a single monitoring system.

### DETAILED DESCRIPTION OF THE DRAWINGS

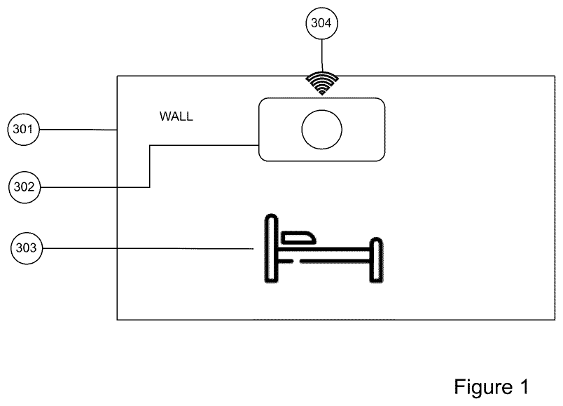

FIG. 1 illustrates an embodiment wherein a wall or stable mountable item (301) is depicted, which serves as a suitable structure for the installation or hanging of a Thermal Health Monitor (THM) camera device (302). The schematic diagram further indicates the presence of a monitoring field of view (303) associated with the THM device, which allows for the observation and assessment of thermal conditions. Additionally, the diagram highlights the incorporation of a Wi-Fi connectivity module (304) within the THM device, enabling seamless wireless communication and data transmission. It is worth noting that the depicted interior space (301) represents a conducive environment for the implementation and deployment of the thermal health monitoring unit.

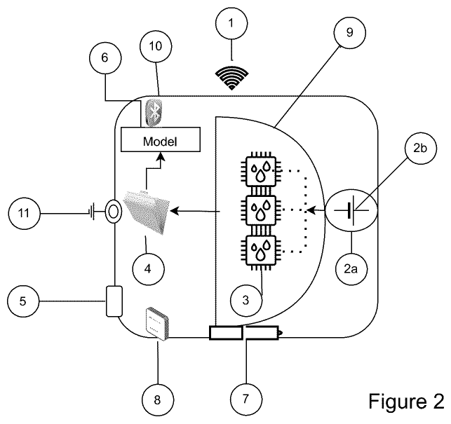

FIG. 2 is a schematic diagram that showcases various components of the device according to an embodiment. The depicted embodiment includes a Wi-Fi connection (1), thereby enabling communication capabilities for the device. The device incorporates a securely mounted thermal camera (2) responsible for capturing and recording thermal data. Furthermore, the device encompasses sensors (3), wherein said sensors are configured to detect abrupt fluctuations in body temperature or movements. These sensors thereby convert the thermal input into a machine-readable JSON file format, facilitating efficient comprehension and predictive modeling. Additionally, a file generator and data aggregator (4) are included to facilitate streamlined data processing and management within the device. Moreover, the device comprises a mobile application interface (5) configured to control the device's operations and functions via a mobile application and provide alert notifications to users. Furthermore, the device incorporates a send alert notification interface (6) configured to transmit generated alerts to medical personnel or other designated recipients, thereby indicating a potential health issue based on analyses of the thermal images. (7) battery backup to ensure uninterrupted operation in case of AC power down or failure. Additionally, it provides a SIM card slot option (8) to enable direct data transmission to healthcare providers, doctors, or for making emergency calls. The device is equipped with an aluminum sensor shield (9) to minimize heat generation. Furthermore, it incorporates Bluetooth technology (10) for seamless connectivity with other devices. Additionally, the device features a USB slot (11) to enhance storage options.

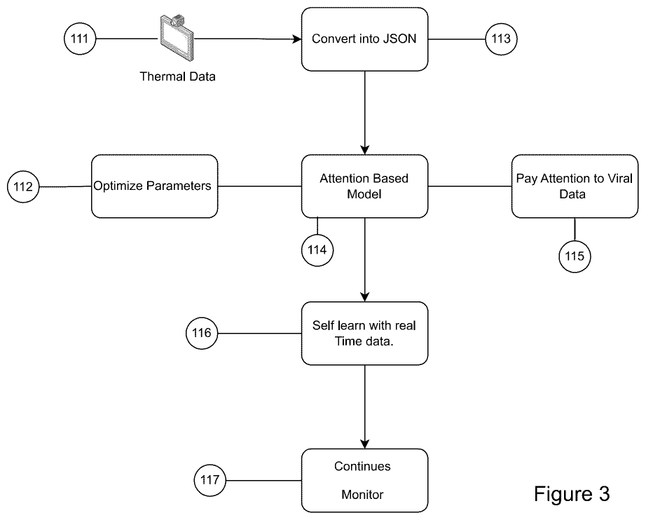

FIG. 3 is an illustration depicting a flowchart that outlines the implementation of thermal camera-based monitoring for capturing a subject's physiological parameters. Thermal camera (111) is employed to non-invasively record and monitor the subject's continuous body temperature. The thermal camera (111) utilized may be a thermographic infrared camera, capable of capturing thermal images that depict variations in body heat across the subject's body. It is positioned strategically to optimize thermal imaging of the subject.

Following the temperature data recording by the thermal camera (111), the system proceeds with data collection and conversion to JSON format (113). JSON (JavaScript Object Notation) is used as a data interchange format, facilitating efficient data transfer and compatibility across various computing platforms and programming languages.

The collected real-time data undergoes parameter optimization (112) within the system. This process involves the application of algorithms and statistical methods to refine and enhance the accuracy and efficiency of data analysis and processing.

An attention-based model (114) is incorporated into the embodiment, which processes the optimized parameters alongside additional subject-related information. The attention-based model (114) employs specialized algorithms and machine learning techniques to evaluate the subject's condition, movements, body temperature, sleep patterns, and relevant viral information. The model's attention mechanisms allow for prioritization and weighting of data inputs, thereby generating actionable information.

The attention-based model (114) utilizes predictive analytics to forecast potential health conditions or abnormalities in the subject. The actionable information generated can include alerts, notifications, or recommendations, which may be communicated to authorized personnel or healthcare professionals for further analysis and intervention.

It is important to note that the attention-based model (114) continuously learns from new data and actively monitors (117) the subject's physiological parameters. This ongoing learning process enables the model to adapt and improve its predictive capabilities over time, enhancing the overall effectiveness of the thermal camera-based monitoring system.

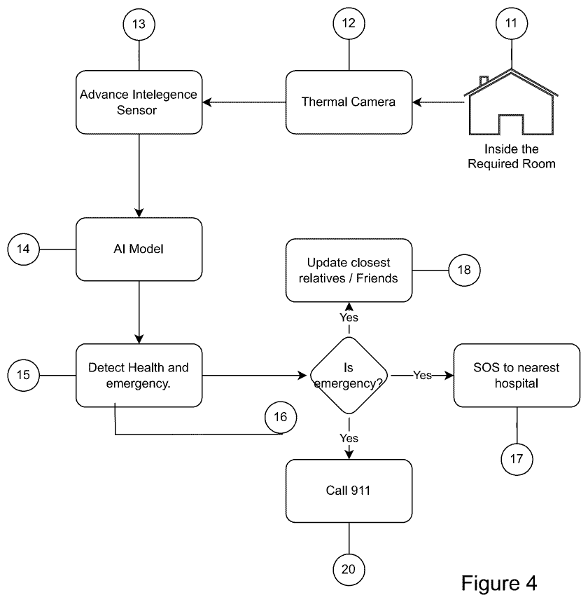

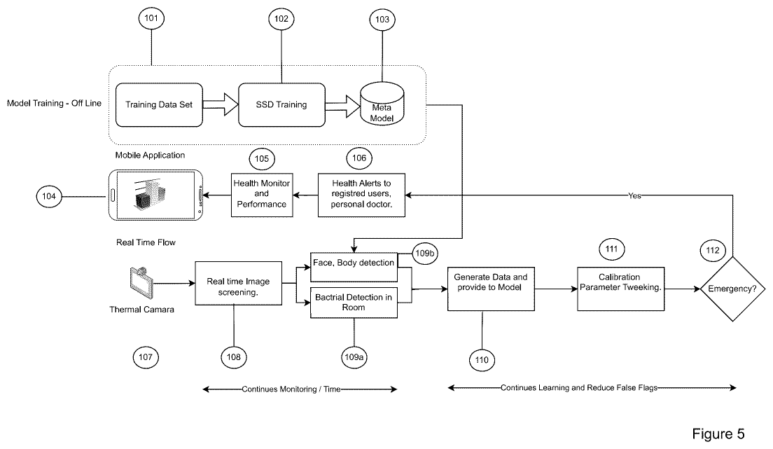

FIG. 4 is a schematic flowchart outlines the operational steps of a monitoring process deployed within a residential area or any specified location. The thermal camera (12) is strategically positioned to capture thermal images and detect variations in temperature. Sensors (13) is employed to effectively capture the visual data transmitted by the thermal camera. The visual data obtained from the thermal camera is then fed into an AI model (14). It evaluates the thermal patterns, identifies anomalies, and determines whether any emergency situation or health concern is present. When an emergency situation or a specific health condition is detected by the AI model, the system initiates a series of actions to ensure prompt assistance. First, notifications are promptly sent to medical personnel, alerting them about the detected emergency situation or health concern. This allows medical professionals to receive timely information and make informed decisions regarding the appropriate course of action (20).

Simultaneously, the system generates SOS alerts (16, 17, 18) that are immediately conveyed to the individuals in need or their caregivers. These alerts can be in the form of visual or auditory signals, ensuring that the affected individuals are made aware of the emergency situation or health concern. The SOS alerts serve as an urgent call for attention and prompt the affected individuals to seek appropriate help.

FIG. 5 is a comprehensive flowchart outlining the operational steps and components involved in the idea. The invention entails the utilization of subject-specific training data sets (101) tailored to individual subject types, thereby enhancing the accuracy and reliability of the detection process. This training data is employed in conjunction with a Single-Shot-Multibox Detector (SSD) (102), a state-of-the-art object detection framework renowned for its real-time detection capabilities.

The system incorporates a trained data model (103) enabling it to effectively identify and classify various health-related factors in real-time. To facilitate seamless access and control over the health monitoring device, a mobile application (104) is integrated into the system. Real-time data acquisition is facilitated by a thermal camera (107), capturing vital physiological information for further analysis. This captured data is subsequently subjected to comprehensive analysis (108) utilizing advanced algorithms and computational techniques. By employing data processing methodologies, the system can detect and identify a range of health conditions, determine probabilities of bacterial presence, and effectively differentiate between subjects. The culmination of analytical processes results in the generation of actionable results (110) that provide insights into the subject's health status. The system incorporates calibration and parameter tweaking mechanisms (111, 112) that fine-tune and optimize the performance of the invention. These procedures allow for the refinement of data analysis techniques, enhancing the reliability of the generated results

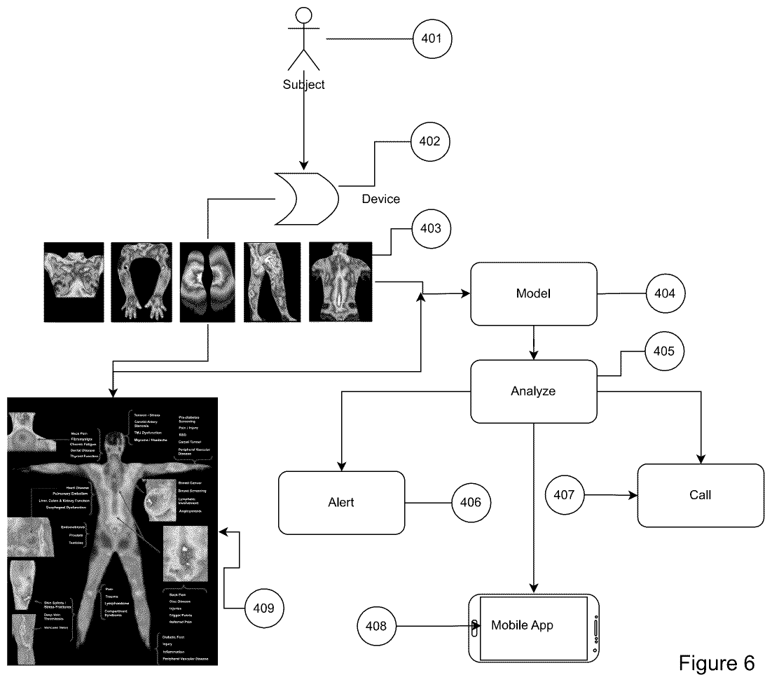

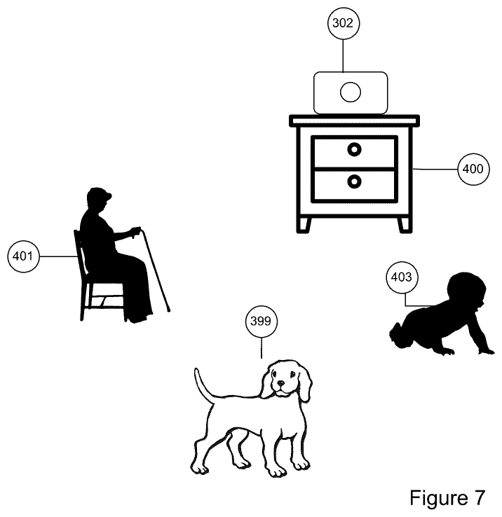

FIG. 6 is a flowchart wherein images are combined with a monitoring process. The embodiment involves the utilization of a health monitor device (402) configured to capture images of a living subject (401) for the purpose of identifying various health conditions and infections (403). The obtained image data is therin processed and provided as input to a model (405), which performs decision-making and facilitates continuous monitoring of the subject's health status. In emergency situations, the notifications initiate emergency calls to doctors or ambulance services (407) to provide timely medical assistance.

FIG. 7 is an embodiment wherein an elderly individual (401), an animal (399), and a child (403) are shown. The present invention involves the placement of a THM camera (302) upon the object (400) for the purpose of monitoring and detecting any abrupt movements or health-related incidents occurring within the body. THM captures and analyzes real-time data in order to provide timely alerts and responses to potential health concerns.

The THM camera can detect subtle temperature variations and patterns on the surface of the object (such as the human body or animal), allowing for the identification of potential anomalies or irregularities. These temperature variations may be indicative of various health conditions, such as inflammation, injury, or physiological changes.

The real-time monitoring and analysis capabilities of the THM camera enable prompt detection and notification of any significant or concerning changes in the monitored subjects. This early warning system ensures that immediate action can be taken to address any potential health issues, allowing for timely medical interventions or appropriate care measures.

### DETAILED DESCRIPTION OF THE INVENTION

The present invention relates to a home health monitoring system that utilizes thermal imaging to detect changes in body temperature and thermal patterns of occupants, such as elderly individuals, children, or pets, as an indicator of their health status and potential emergencies requiring a timely response.

The system comprises a thermal camera installed in the home environment with a view of the monitored area. The thermal camera continuously captures thermal images and transmits the data wirelessly to a processing unit for analysis.

The processing unit includes a machine learning model trained on representative thermal data to learn how to identify abnormalities indicative of potential health issues or emergencies. When anomalies are detected in the thermal images of an occupant, an alert is automatically generated and transmitted to designated contacts such as family members, caretakers or emergency services.

The alerts contain details about the thermal anomaly detected, its likely cause, and recommended actions. They are transmitted through various channels such as text messages, phone calls, and mobile applications, to ensure timely receipt by the designated contacts.

The machine learning model is continuously refined and improved through exposure to additional thermal data, allowing it to become more sensitive and accurate at anomaly detection over time. This ongoing optimization aims to maximize the health monitoring benefits provided by the system.

THM has the capability to detect and visualize the heat patterns emitted by both individuals and pets. By capturing and interpreting the infrared radiation emitted by living beings, thermal cameras can provide valuable insights into temperature variations and hotspots, enabling various applications in fields such as healthcare, safety, and animal care.

The system is especially beneficial for monitoring vulnerable populations such as elderly individuals, children, and pets. It can detect health events such as falls, fainting spells, seizures and illnesses at an early stage, facilitating rapid response to reduce health consequences and mitigate issues.

The integration of thermal imaging and machine learning technologies allows the system to provide comprehensive, around-the-clock health monitoring with a high degree of accuracy and responsiveness within home environments. The home health monitoring system has a variety of applications, including elderly care, children's health monitoring, pet monitoring, and remote patient monitoring. By leveraging the capabilities of thermal imaging and machine learning technologies, the system aims to provide a comprehensive, proactive, and adaptable health monitoring solution within home environments.

1. A home health monitoring system comprising:
a thermal camera installed in a home environment configured to capture thermal images of individuals or pets within its field of view;
a machine learning model trained on synthetic and real thermal data for detecting and analyzing variations in body temperature and thermal patterns from said thermal images, and for identifying thermal anomalies indicative of potential health issues;
an alert system for generating alerts when said machine learning model detects a thermal anomaly, said alerts comprising notifications to nearby hospitals, care facilities, and designated family and friends;
wherein said system enables timely medical response and intervention through rapid detection of and alerting regarding said thermal anomalies.
The system of claim 1, wherein said potential health issues comprise sudden falls, collapses, or medical emergencies.
The system of claim 1, wherein said alerts comprise reports containing details of said thermal anomaly and said individuals or pets.
The system of claim 1, further comprising a reporting system for generating detailed reports of thermal data and activities over time of said individuals or pets to provide health insights.
The system of claim 1, wherein said machine learning model is configured to improve over time through continued exposure to additional synthetic and real thermal data.
